(19) United States
(12) Patent Application Publication
(10) Pub. No.: US 2025/0340185 A1
(43) Pub. Date: Nov. 6, 2025

(54) METHOD OF CONTROLLING A VEHICLE INTERIOR STERILIZATION APPARATUS

(71) Applicants: HYUNDAI MOTOR COMPANY, Seoul (KR); KIA CORPORATION, Seoul (KR); SL Corporation, Daegu (KR)

(72) Inventors: Jae Seung Lee, Hwaseong-si (KR); Hyun Woo Choi, Daegu (KR); Kwang Soo Park, Daegu (KR); Myeong Joo Seo, Gyeongsan-si (KR); Ji Hoon Yang, Gyeongsan-si (KR); Tae Seok Seo, Gyeongsan-si (KR); Seung Yeop Lee, Gyeongsan-si (KR)

(73) Assignees: HYUNDAI MOTOR COMPANY, Seoul (KR); KIA CORPORATION, Seoul (KR); SL Corporation, Daegu (KR)

(21) Appl. No.: 18/929,237

(22) Filed: Oct. 28, 2024

(30) Foreign Application Priority Data
May 3, 2024 (KR) 10-2024-0059309

(51) Int. Cl. B60S 1/64; A61L 9/20; B60R 16/033

(52) U.S. Cl. CPC B60S 1/64; A61L 9/20; B60R 16/033; A61L 2202/14; A61L 2202/16

(57) ABSTRACT

A method of controlling a vehicle interior sterilization apparatus includes determining, by the control unit, whether a sterilization precondition for a vehicle is satisfied and a user sterilization request signal is received; determining, by the control unit, when the sterilization precondition is satisfied and the sterilization request signal is received, whether a sterilization stability condition is satisfied; determining, by the control unit, when the sterilization stability condition is satisfied, whether a battery operation condition is satisfied; and performing, vehicle interior sterilization using the vehicle interior sterilization apparatus, when the battery operation condition is satisfied. The performing the vehicle interior sterilization includes opening a cover unit positioned to cover an opening of a housing provided inside a roof lining and turning on a light source unit exposed to the interior of the vehicle.

### CROSS-REFERENCE TO RELATED APPLICATION

This application claims, under 35 U.S.C. § 119(a), the benefit of and priority to Korean Patent Application No. 10-2024-0059309 filed on May 3, 2024, the entire contents of which are incorporated herein by reference.

### BACKGROUND

### (a) Technical Field

The present disclosure relates to a method of controlling a vehicle interior sterilization apparatus, and more particularly, to a method of controlling a vehicle interior sterilization apparatus, capable of sterilizing the interior of a vehicle using a light source unit by selectively opening a cover unit of the sterilization apparatus that is provided inside a roof lining.

### (b) Background Art

In general, in order to maintain a vehicle's interior air environment in a comfortable or suitable condition for a driver, a vehicle air conditioning system has functions such as dehumidification, humidification, purification, ventilation, and aromatization, and steady temperature and humidity functions for uniformly keeping temperature and humidity, in addition to main functions of cooling and heating.

In such a vehicle air conditioning system, air flows into a vehicle through a cabin filter that filters and purifies (e.g., removes) pollutants such as exhaust gas, dust, and pollen in the air. Accordingly, the cabin filter plays an important role in ensuring a comfortable interior environment.

However, since the cabin filter is usually located inside a glove box in front of a passenger seat, in order to inspect or replace the cabin filter, a driver must open the globe box, separate the cabin filter, and then re-insert the cabin filter. Further, since the driver must install the cabin filter in an air flowing direction, that is, in a correct direction, it is very laborious to frequently replace the cabin filter. In addition, it is difficult to immediately check a contamination state, and thus, it takes quite a long time to replace the cabin filter.

Furthermore, it is difficult to sterilize germs contaminated on the surface of the interior of the vehicle using existing air conditioning systems having an air purifying function.

The above information disclosed in this Background section is provided only to enhance understanding of the background of the disclosure. Therefore, the Background section may contain information that does not form the prior art that is already known in this country to a person of ordinary skill in the art.

### SUMMARY OF THE DISCLOSURE

The present disclosure has been made in an effort to solve the above-described problems associated with prior art. An object of the present disclosure is to provide a method of controlling a vehicle interior sterilization apparatus for performing interior sterilization of the vehicle in a case where (e.g., when) user safety is ensured using the sterilization apparatus provided inside a roof lining.

Another object of the present disclosure is to provide a method of controlling a vehicle interior sterilization apparatus capable of determining the presence or absence of a passenger to perform (e.g., before performing) sterilization, using a cover unit that is selectively opened or closed.

The objects of the present disclosure are not limited to the objects mentioned above. Other objects of the present disclosure that are not mentioned above can be understood from the following description and can be more clearly understood from the examples of the present disclosure. Further, the objects of the present disclosure may be realized by means and combinations thereof as indicated in the claims.

In one aspect, the present disclosure provides a method of controlling a vehicle interior sterilization apparatus, including: determining, by a control unit, whether a sterilization precondition for a vehicle is satisfied and a user sterilization request signal is received; determining, by the control unit, whether a sterilization stability condition is satisfied, when the sterilization precondition is satisfied and the sterilization request signal is received; determining, by the control unit, when the sterilization stability condition is satisfied, whether a battery operation condition is satisfied; and performing, when the battery operation condition is satisfied, vehicle interior sterilization using the sterilization apparatus. The performing the vehicle interior sterilization including opening a cover unit positioned to selectively cover an opening of a housing provided inside a roof lining, and turning on a light source unit exposed to the interior of the vehicle.

In an embodiment, the determining whether the sterilization precondition is satisfied may include determining whether the vehicle is in an ignition-off state and a door is in a locked state after a passenger exits the vehicle, by the control unit.

In another embodiment, determining whether the sterilization stability condition is satisfied may include determining that the sterilization stability condition is satisfied, when the following conditions are satisfied: the vehicle is stopped, a gear parking input is received, a window of the vehicle is closed, a door of the vehicle is locked, a smart key of the vehicle is outside, seat weight is not detected, and no object is detected in a rear seat of the vehicle, by the control unit.

In still another embodiment, the determining whether the sterilization stability condition is satisfied may further include terminating an operation in a case where the sterilization stability condition is not satisfied.

In yet another embodiment, the determining whether the battery operation condition is satisfied may include comparing a state of charge of a battery with a set value.

In still yet another embodiment, the comparing the state of charge of the battery with the set value, may include terminating an operation of the sterilization apparatus, when the state of charge of the battery is equal to or lower than a first set value, and setting an output-limit sterilization mode of the sterilization apparatus, when the state of charge of the battery is greater than the first set value and is equal to or lower than a second set value.

In a further embodiment, the performing the vehicle interior sterilization using the sterilization apparatus, may include performing control, when an interior temperature of the vehicle exceeds a first set temperature, for reducing an operating time of the sterilization apparatus or the amount of current applied to the light source unit, by the control unit.

In another further embodiment, the performing the vehicle interior sterilization using the sterilization apparatus, may include controlling the amount of current applied from the battery to each of the plurality of LEDs that form the light source unit, or controlling the number of lighting times of each of the plurality of LEDs, by the control unit.

In still another further embodiment, the control unit may perform control for reducing the current applied to each of the LEDs positioned adjacent to the sterilization apparatus and increasing the current applied to each of the LEDs that emits light of the interior of the vehicle positioned relatively far from the sterilization apparatus.

In still yet another further embodiment, in the determining whether the sterilization precondition for the vehicle is satisfied and the user sterilization request signal is received, the control unit may determine, in a case where the sterilization precondition is satisfied, whether the user sterilization request signal is received through external manipulation unit.

Other aspects and embodiments of the disclosure are discussed infra.

### BRIEF DESCRIPTION OF THE DRAWINGS

The above and other features of the present disclosure are described in detail with reference to several embodiments thereof illustrated in the accompanying drawings which are given hereinbelow by way of illustration only, and thus do not limit the present disclosure, and wherein:

FIG. 1 is a diagram showing a vehicle interior sterilization apparatus located on a roof lining according to an embodiment of the present disclosure;

FIG. 2 is a perspective view of a vehicle interior sterilization apparatus according to an embodiment of the present disclosure;

FIG. 3 is a side cross-sectional view of a vehicle interior sterilization apparatus according to an embodiment of the present disclosure;

FIG. 4 is a rear view of a light source unit of a vehicle interior sterilization apparatus according to an embodiment of the present disclosure;

FIG. 5A is a diagram showing a rotational structure of a cover unit of a vehicle interior sterilization apparatus according to an embodiment of the present disclosure;

FIG. 5B is a diagram showing a fastening structure between a cover unit and an actuator unit in a vehicle interior sterilization apparatus according to an embodiment of the present disclosure;

FIG. 6 is a flowchart showing a method of controlling a vehicle interior sterilization apparatus through an interior manipulation unit, according to an embodiment of the present disclosure; and

FIG. 7 is a flowchart showing a method of controlling a vehicle interior sterilization apparatus using an outdoor manipulation unit, according to another embodiment of the present disclosure.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes may be determined in part by the particular intended application and use environment.

In the figures, reference numbers refer to the same or equivalent parts of the present disclosure throughout the several figures of the drawing.

### DETAILED DESCRIPTION

Hereinafter, reference is made in detail to various embodiments of the present disclosure, examples of which are illustrated in the accompanying drawings and described below. While the disclosure is described in conjunction with several embodiments, it should be understood that the present description is not intended to limit the disclosure to the embodiments described herein. On the contrary, the disclosure is intended to cover not only the embodiments described herein, but also various alternatives, modifications, equivalents, and other embodiments, within the spirit and scope of the disclosure as defined by the appended claims.

In the specification, terms such as “ . . . unit”, “ . . . module”, and the like refer to a unit that performs at least one function or operation, and may be implemented by hardware, software, or a combination thereof.

Further, the terms used in the specification are merely used to describe specific embodiments and are not intended to limit the disclosure to the embodiments. As used herein, the singular form “a/an” is intended to include the plural forms as well, unless the context clearly indicates otherwise.

In addition, the terms “first”, “second”, etc. used to describe various elements are merely used to distinguish one element from another, and do not limit the order of the elements. For example, a first element may be termed a second element, and similarly, a second element may be termed a first element, without departing from the scope of the embodiments of the present disclosure.

The following examples illustrate the disclosure and are not intended to limit the same. When a component, device, element, or the like of the present disclosure is described as having a purpose or performing an operation, function, or the like, the component, device, or element should be considered herein as being “configured to” meet that purpose or perform that operation or function.

A control unit 600 may include a memory that stores data on an algorithm for controlling operations of various components provided in a vehicle or a program for the algorithm, and a processor that performs the above-described operations (e.g., algorithm) using the data stored in the memory. Here, the memory and the processor may be implemented as separate chips, respectively. Alternatively, the memory and the processor may be implemented as a single chip. For example, the control unit 600 may include at least one of an electronic control unit (ECU), a central processing unit (CPU), a microprocessor unit (MPU), a microcontroller unit (MCU), an application processor (AP), or any other processor known in the art. Further, the control unit 600 may include a combination of software and hardware capable of performing operations related to at least one application or program for executing methods according to the embodiments of the present disclosure.

Hereinafter, embodiments of the present disclosure are described in detail with reference to the accompanying drawings. In the description, identical or corresponding components are assigned the same reference numbers and repetitive or duplicative descriptions thereof have been omitted.

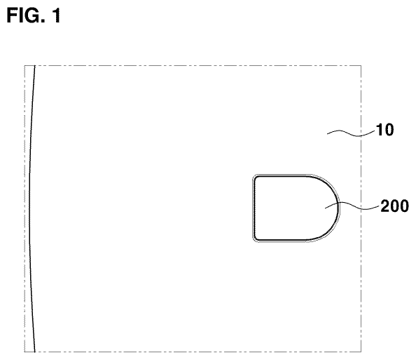

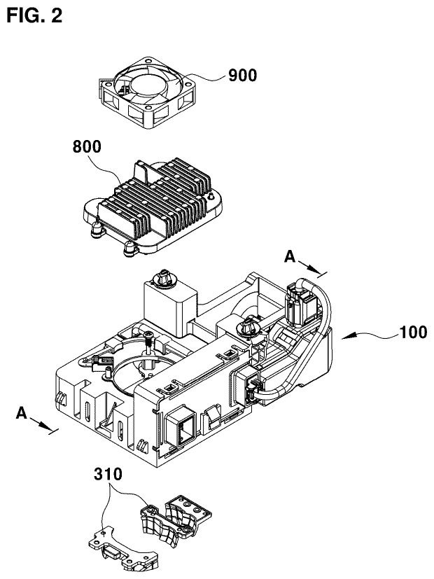

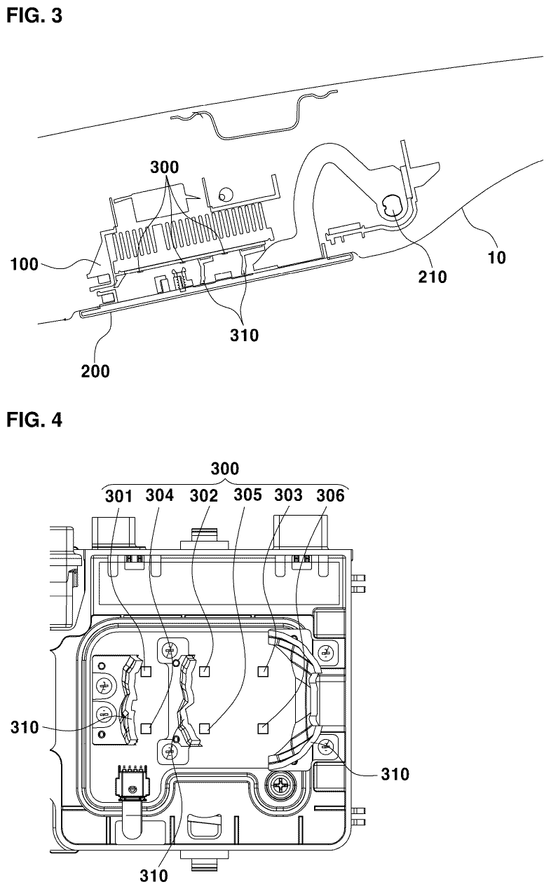

FIG. 1 shows one side of a roof lining 10 according to a vehicle interior sterilization apparatus according to an embodiment of the present disclosure. FIG. 2 shows a configuration of the vehicle interior sterilization apparatus. FIG. 3 shows a side cross-sectional view of an area AA of FIG. 2.

As shown in the figures, the roof lining 10 is positioned on an upper surface of the vehicle's interior and includes a vehicle interior sterilization apparatus. The vehicle interior sterilization apparatus has a configuration in which a housing 100 is inserted inside the roof lining 10 to form the same plane as (e.g., so as to be flush with) the roof lining 10, and a cover unit 200 is combined with the housing 100 to form the same plane as (e.g., so as to be flush with) the roof lining 10. The cover unit 200 may be made of the same material as that of the roof lining 10 to provide a sense of unity with the roof lining 10.

Here, the cover unit 200 is combined with or coupled to one end portion of the housing 100 with reference to a shaft 210. The cover unit is configured to be selectively opened (e.g., and closed) by rotating around the shaft 210. One surface of the housing 100 facing the cover unit 200 is formed with an opening, and a light source unit 300 is provided inside the opening.

The light source unit 300 may include at least one LED, and may include an ultraviolet LED and a visible LED. As an example, the light source unit 300 may include an ultraviolet LED that emits ultraviolet light, a visible light LED that emits visible light, and a control unit 600 that selects light and controls the emission of each LED (e.g., the ultraviolet LED and the visible LED).

The ultraviolet LED may emit short-wavelength light in the range of 10 nm to 380 nm wavelength, or more narrowly, ultraviolet light in the range of 200 nm to 380 nm wavelength. Here, the ultraviolet LED may emit light in the UV-A region (e.g., range, spectrum) or UV-C region (e.g., range, spectruc) to sterilize an object (e.g., disposed in an interior of the vehicle). The ultraviolet LEDs may be manufactured using nitride-based compound semiconductors such as GaN, AlGaN, InGaN, and InAlGaN.

Further, the vehicle interior sterilization apparatus further includes a reflector 310 that is provided adjacent to the light source unit 300 (e.g., light source 300) to reflect light emitted from the light source unit 300. The reflector 310 may be configured so that a wider area of the interior area can be irradiated than an area directly irradiated by the light source unit 300 (e.g., so as to increase the size of an area irradiated by the light source unit 300.

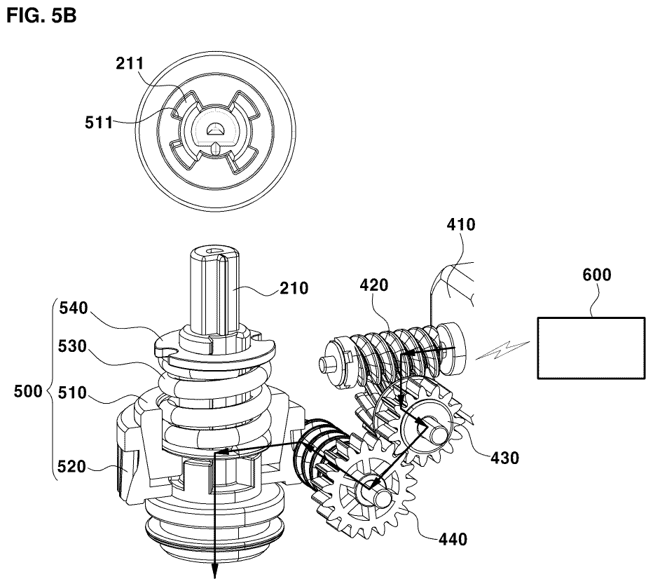

The shaft 210 of the cover unit 200 is coupled to an actuator unit 400 (e.g., actuator 400) positioned on the roof lining 10 adjacent to the housing 100, so that a driving force of the actuator unit 400 is applied to one end portion of the cover unit 200 (see FIG. 5A).

As an example, the actuator unit 400 may include a motor 410 that supplies driving force under the control of the control unit 600, a first worm gear 420 provided on an output shaft of the motor 410, a second worm gear 440 provided in a clutch unit 500 disposed outside the shaft 210, and a worm wheel gear 430 provided between the first worm gear 420 and the second worm gear 440 (see FIG. 5B).

Here, the control unit 600 controls the operation of the motor 410 to apply rotational force to the clutch unit 500, so that the cover unit 200 rotates around the shaft 210 to be opened.

Here, the cover unit 200 may include a plurality of plates, and the actuator unit 400 may be disposed on the rear surface of the cover unit 200 and may provide a driving force to move the cover unit 200 in one direction so that the cover unit 200 is wound along the outside of the housing 100.

In addition, a heat sink 800 may be provided inside the housing 100 on the opposite side of the light source unit 300, and a cooling fan 900 may be provided above the heat sink 800. This configuration performs a function of dispersing heat generated by the light source unit 300 while sterilizing the interior of the vehicle using the light source unit 300.

The control unit 600 may control power suppled to the actuator unit 400 The control unit 600 may determine whether there is an occupant in the vehicle. Further, the control unit 600 may receive a user's request for sterilization, and in response, may control the opening of the cover unit 200 and the emission or irradiation of light from the light source unit 300. According to an embodiment of the present disclosure, the control unit 600 may determine the presence or absence of an occupant using an interior camera, a seat position sensor, and the like. The control unit 600 may perform control, in a case where (e.g., when) it is determined that there is no occupant, so that the cover unit 200 is opened and the light source unit 300 emits light.

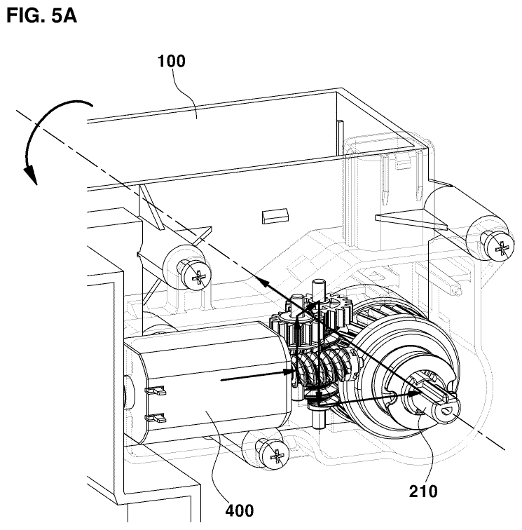

FIG. 4 shows a configuration of the light source unit 300 and the reflector 310 positioned inside the housing 100.

As shown in FIG. 4, for example, the light source unit 300 may have a configuration in which six light sources 301, 302, 303, 304, 305, 306 are arranged in two columns and three rows. Further, the reflector 310 has three reflector members, in which two of the reflector members are positioned at both end portions and one reflector member is positioned between the two reflector members. The reflector 310 may be positioned to disperse light emitted from the light source unit 300 over a wider area.

Furthermore, the reflector 310 is positioned to minimize uneven light energy according to an installation height inside the vehicle. The light intensity of the light source unit 300 may be controlled to suppress excessive light energy applied to areas relatively close to the light source unit 300 positioned on the roof, such as a console or door handles located inside the vehicle, or the position of the reflector 310 may be adjusted to minimize the light energy. In addition, the light intensity of the light source unit 300 may be controlled to increase light energy applied to a relatively dark area that sufficient light does not reach, such as a seat side area, or the position of the reflector 310 may be adjusted so that the dark area is irradiated with the reflected light.

Accordingly, the angle, shape, and position of the reflector 310 may be set according to interior conditions of the vehicle.

FIGS. 5A and 5B show a coupling relationship between the shaft 210 of the cover unit 200 and the actuator unit 400, according to an embodiment of the present disclosure.

The actuator unit 400 is positioned on the side of the housing 100 or inside the roof lining 10 and is coupled to the shaft 210 of the cover unit 200. Specifically, the actuator unit 400 includes the first worm gear 420 positioned on the output shaft of the motor 410, the second worm gear 440 engaged with the clutch unit 500 (e.g., clutch 500) outside the shaft 210, and the worm wheel gear 430 positioned between the first worm gear 420 and the second worm gear 440.

In a case where a request to open the cover unit 200 is received, the control unit 600 supplies power to the motor 410, and accordingly, the first worm gear 420 positioned on the output shaft of the motor 410 is rotated. The rotational force of the first worm gear 420 is transmitted to the second worm gear 440 through the worm wheel gear 430. A clutch gear 520 of the clutch unit 500, which is engaged with the second worm gear 440, is rotated integrally with the shaft 210 by the rotational force of the second worm gear 440.

Here, the clutch gear 520 may be configured to rotate integrally with a clutch cylinder 510 fixed to the outer peripheral surface of the shaft 210 when rotating in one direction and to slip on the clutch cylinder 510 when rotating in the other direction. In this case, in response to the request to open the cover unit 200, the clutch gear 520 is engaged with the clutch cylinder 510 and rotates integrally with the shaft 210 in the opening direction of the cover unit 200. Further, the control unit 600 is configured to rotate the motor 410 to close the cover unit 200 after the sterilization is completed. Here, the clutch cylinder 510 and the clutch gear 520 may be engaged with each other by a frictional force. Therefore, in a case where the clutch gear 520 receives the rotational force from the second worm gear 440, the clutch cylinder 510 and the clutch gear 520 are integrally rotated along the opening direction of the cover unit 200 by friction between the clutch cylinder 510 and the clutch gear 520.

In this case, in performing sterilization, in a case where (e.g., when) there is failure of the sterilization apparatus, such as battery discharge or drive failure, the cover unit 200 is not closed. Then, in a case where the rotational force is manually applied to the cover unit 200 in the closing direction of the cover unit 200, the clutch cylinder 510 slips on the clutch gear 520, so that the shaft 210 and the clutch cylinder 510 may be rotated independently of the clutch gear 520.

In a case where the clutch cylinder 510 is rotated integrally with the shaft 210 in the other direction through a manual operation, an overriding force greater than the frictional force between the clutch cylinder 510 and the clutch gear 520 is provided, so that the clutch cylinder 510 may be rotated independently of the clutch gear 520. In this case, the rotational force of the clutch cylinder 510 is not transmitted to the clutch gear 520.

In summary, the clutch gear 520 and the clutch cylinder 510 are integrally rotated in response to the rotation in the opening direction of the cover unit 200 during sterilization, and then, the clutch gear 520 and the clutch cylinder 510 are integrally rotated in the other direction after sterilization.

However, in a case where the cover unit 200 is in the open state due to failure of the sterilization apparatus, and in a case where the rotational force is applied in the closing direction of the cover unit 200 according to manual operation, the clutch gear 520 and the clutch cylinder 510 may be moved independently of each other.

To this end, as described above, the actuator unit 400 includes the motor 410, and the control unit 600 applies power to the motor 410 in response to the opening and closing requests of the cover unit 200. In a case where a user manually operates the cover unit 200 in the closing direction, the clutch cylinder 510 is rotated integrally with the shaft 210 independently of the clutch gear 520, so that the cover unit 200 is moved to the closed position.

The clutch cylinder 510 includes a groove part 511 that is engaged with the shaft 210, and the shaft 210 includes an engaging part 211 that is inserted inside the groove part 511. Here, the engaging part 211 of the shaft 210 is configured to face one end portion of the clutch cylinder 510.

In addition, a stopper 540 is provided at one end portion of the shaft 210, and a spring 530 is provided between the clutch cylinder 510 and the stopper 540. The spring 530 provides elastic force between the stopper 540 positioned at one end portion of the shaft 210 and the clutch cylinder 510 to determine the frictional force applied between the clutch cylinder 510 and the clutch gear 520.

Accordingly, in a case where the cover unit 200 is manually operated to be closed, a slip force between the clutch gear 520 and the clutch cylinder 510 may be set by the elastic force of the spring 530.

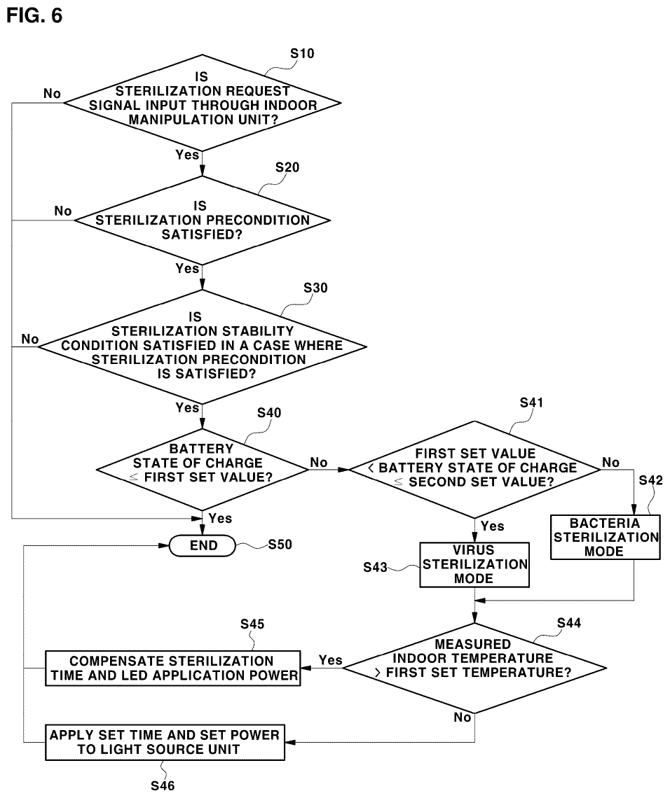

FIG. 6 is a flowchart showing a method of controlling a vehicle interior sterilization apparatus in performing in-vehicle sterilization input through an interior manipulation unit, according to an embodiment of the present disclosure.

As shown in FIG. 6, the control unit determines whether a user's sterilization request signal is input through the interior manipulation unit (S10). The interior manipulation unit may include a dashboard or buttons inside the vehicle and may include an automotive multimedia device provided with AVN (Audio, Video, and Navigation).

A user may input a sterilization request signal through the interior manipulation unit in a state where the user is in the vehicle, and then, the control unit determines whether a sterilization precondition is satisfied. In determining whether the sterilization precondition is satisfied, the control unit may determine whether the vehicle is in an ignition-off state and doors are in a locked state after the user exits the vehicle (S20).

In order to determine whether the sterilization precondition is satisfied, the control unit determines whether the user exits the vehicle and determines whether the vehicle is in the stopped state and the doors are in the locked state.

In a case where it is determined that the sterilization precondition is satisfied, the control unit determines whether a sterilization stability condition is satisfied. The sterilization stability condition refers to a condition for sterilizing the interior of the vehicle. Here, in determining whether the sterilization stability condition is satisfied, the control unit determines whether specific conditions of vehicle stop, gear parking input, window closed, door locked, smart key outside, seat weight not detected, and rear seat object not detected are all satisfied. In a case where at least one of the specific conditions is not satisfied, the control unit terminates the operation of the sterilization apparatus (S30).

In this way, the sterilization stability condition is determined in consideration of whether there is an object in the vehicle and whether light for sterilization is exposed through a window. In addition, the control unit may re-determine whether a person or an object is in the vehicle, thereby making it possible to prevent light of a light source unit from being emitted on to the person or object.

Here, the determination of the sterilization precondition and the determination of the sterilization stability condition are performed in a temporal order from a starting point at which the sterilization request is received. The determination of the sterilization precondition is performed by determining whether an occupant exits the vehicle before the sterilization request. The determination of the sterilization stability condition is performed by determining whether a user or an object exists inside the vehicle at a point at which the sterilization is performed.

Then, the control unit determines whether a battery operation condition is satisfied, and in a case where it is determined that a state of charge of a battery is sufficient, the control unit sets an interior sterilization mode through the sterilization apparatus. Specifically, in a case where the measured state of charge of the battery is equal to or lower than a first set value (S40), the control unit terminates the operation of the sterilization apparatus (S50), and in a case where the measured state of charge of the battery is greater than the first set value and is equal to or lower than a second set value (S41), the control unit limits an output of the sterilization apparatus to set the interior sterilization (S43). As a method of limiting the output of the sterilization apparatus, the control unit may perform control for reducing the amount of power supplied to the light source unit 300, reducing a set number of lighting times of the light source unit 300, or reducing an operating time of the light source unit 300. Here, the case in which the output of the sterilization apparatus is limited may be referred to as a virus sterilization mode. Additionally, in a case where the state of charge of the battery exceeds the second set value (S41), the control unit performs a bacteria sterilization mode of the sterilization apparatus (S42).

As shown in FIGS. 1-5B, the sterilization apparatus includes the housing 100 that is inserted in the roof lining 10, and the cover unit 200 configured to selectively open and close the opening of the housing 100. In performing interior sterilization using the sterilization apparatus, the control unit performs control for opening the cover unit 200 by applying the driving force of the actuator unit, so that the light source unit 300 positioned inside the cover unit 200 faces the interior of the vehicle.

Further, after setting the interior sterilization mode to the virus sterilization mode or bacteria sterilization mode by the sterilization apparatus, the control unit receives interior temperature information from a sensor unit provided in the vehicle.

In a case where the measured interior temperature exceeds a first set temperature (S44), the control unit may perform compensation control for lowering the output of the light source unit 300 of the sterilization apparatus and may extend the operating time of the light source unit 300 (S45). In a case where the interior temperature exceeds the first set temperature, the control unit performs control for reducing the output power applied to the light source unit or reducing the number of lighting times of the light source unit 300 to prevent the interior temperature of the vehicle from rising excessively (S45). Here, in a case where the interior temperature exceeds the first set temperature, the control unit may perform compensation control for reducing the power applied to the light source unit 300 and simultaneously or separately reducing the intensity of light of the light source units 300.

More specifically, in a case where the interior temperature exceeds the first set temperature, the control unit performs compensation control for reducing current applied from the battery to each of the plurality of LEDs that form the light source unit 300 or performs control for reducing the number of lighting times of each LED.

On the contrary, in a case where the interior temperature is equal to or lower than the first set temperature (S44), the control unit may perform control so that the light source unit 300 is operated with set power, a set number of lighting times, and a set operating time (S46).

Here, in the virus sterilization mode, the light source unit is controlled to perform sterilization at a relatively low state of charge of the battery, in which power is supplied to the light source unit for a relatively short period of time compared with the bacteria sterilization mode.

In one embodiment of the present disclosure, the light source unit may be turned on for 15 minutes in the virus sterilization mode and may be turned on for 60 minutes in the bacteria sterilization mode.

In addition, the control unit may store structural characteristics of the interior of the vehicle. The stored structural characteristics of the interior of the vehicle may include topographical characteristics in a height or vertical direction of the interior of the vehicle. For example, the LEDs may be positioned to minimize uneven light energy according to the heights of parts disposed inside the vehicle. One or more of the LEDs are controlled to reduce excessive light energy applied to areas relatively close to the light source unit 300 located on the roof, such as a console or door handles disposed inside the vehicle. In addition, in order to allow light to be emitted to a dark area that sufficient light does not reach, such as a seat side area, the intensity of light and the number of lighting times of at least one LED corresponding to the area may be controlled.

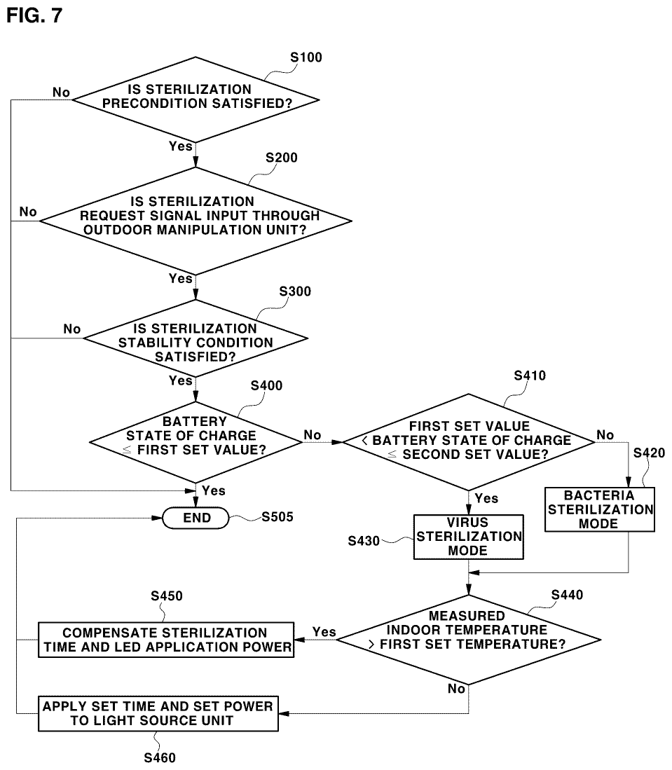

FIG. 7 is a flowchart showing a method of controlling a vehicle interior sterilization apparatus, for inputting a sterilization request to the sterilization apparatus using an external manipulation unit, according to another embodiment of the present disclosure.

The control unit first determines whether a sterilization precondition is satisfied (S100), and then, determines whether a sterilization request signal is received through the external manipulation unit located outside the vehicle (S200).

In determining whether the sterilization precondition is satisfied, the control unit determines whether the vehicle is in an ignition-off state and doors are in a locked state after an occupant exits the vehicle.

In the present embodiment, the external manipulation unit may include a portable communication device, a wireless key, a smartphone application, etc., and may include any smart device capable of remotely communicating with the vehicle.

In a case where the sterilization request signal is received, the control unit determines whether a sterilization stability condition of the vehicle is satisfied. In determining whether the sterilization stability condition is satisfied, the control unit determines whether conditions for sterilizing the interior of the vehicle are satisfied. For example, in determining whether the sterilization stability condition is satisfied, the control unit may determine whether specific conditions of vehicle stop, gear parking input, window closed, door locked, smart key outside, seat weight not detected, and rear seat object not detected are all satisfied. In a case where at least one of the specific conditions is not satisfied, the control unit terminates the operation of the sterilization apparatus (S300).

In determining the sterilization stability condition, the control unit may determine whether there is an object located in the vehicle and whether light for sterilization is exposed through a window. In addition, since the control unit re-determines whether a person or an object is in the vehicle, it is possible to prevent light of a light source unit from being emitted to the person or object.

Referring to FIG. 6, the control unit compares the battery operation condition, that is, the state of charge of the battery with a set value, and then, sequentially performs the steps for interior sterilization.

More specifically, in a case where it is determined that the state of charge of the battery is sufficient, the control unit performs the interior sterilization using the sterilization apparatus (S420). In a case where the measured state of charge of the battery is equal to or lower than the first set value (S400), the control unit terminates the operation of the sterilization apparatus (S505), and in a case where the measured state of charge of the battery is greater than the first set value and is equal to or lower than the second set value (S410), the control unit performs the interior sterilization by limiting the output of the sterilization apparatus (S430). In addition, in a case where the state of charge of the battery exceeds the first set value and is equal to or lower than the second set value, the control unit performs the virus sterilization mode (S430).

In a case where the state of charge of the battery exceeds the second set value, the control unit performs the bacteria sterilization mode (S420). Here, the virus sterilization mode may be set so that the total energy applied to the light source unit is small compared with the bacteria sterilization mode. In one embodiment of the present disclosure, the bacteria sterilization mode may be set so that power is applied to the light source unit for 60 minutes, and the virus sterilization mode may be set so that power is applied to the light source unit for 15 minutes.

In order to execute the sterilization mode, the sterilization apparatus includes the housing 100 that is inserted in the roof lining 10, and the cover unit 200 that selectively opens and closes the opening of the housing 100. In performing the interior sterilization using the sterilization apparatus, the control unit performs control for opening the cover unit 200 by applying the driving force of the actuator unit, so that the light source unit 300 positioned inside the cover unit 200 faces the interior of the vehicle.

After the sterilization mode is set, the control unit determines whether the interior temperature of the vehicle exceeds the first set temperature (S440). In a case where it is determined that the interior temperature of the vehicle exceeds the first set temperature, the control unit performs compensation control for lowering the output of the light source unit 300 of the sterilization apparatus, and/or performs compensation control for allowing the light source unit 300 to be operated for an operating time shorter than a set sterilization time. In a case where the measured interior temperature exceeds the first set temperature, the control unit performs control for reducing the output power applied to the light source unit or reducing the number of lighting times of the light source unit 300 so that the interior temperature of the vehicle does not rise excessively (S450).

In contrast, in a case where the measured interior temperature is equal to or lower than the first set temperature (S440), the control unit performs control for operating the light source unit 300 at set power and a set number of lighting times (S460).

According to the present disclosure, it is possible to maintain a hygienic vehicle by providing the method of controlling the vehicle interior sterilization apparatus through determination processes capable of ensuring user safety in performing sterilization of contaminated areas on the interior surface of the vehicle.

In addition, it is possible to achieve efficient interior sterilization corresponding to the structure of the vehicle by performing control of lighting of the light source unit corresponding to the interior space structure of the vehicle.

The above description merely illustrates several embodiments of the present disclosure. Additionally, the above description is intended to illustrate several embodiments of the present disclosure, and the present disclosure may be applied to various other combinations, modifications, and environments. It should be obvious to those of ordinary skill in the art that changes or modifications may be made to the embodiments described herein and fall within the scope of the inventive concept disclosed in this specification, and equivalents thereto are also within the technical scope of the inventive concept described herein. The above embodiments merely illustrate several examples for implementing the technical idea of the present disclosure, and various modifications for specific applications of the inventive concept are also possible. Accordingly, the above description is not intended to limit the inventive concept to the disclosed embodiments. In addition, the appended claims should be construed to include other equivalent embodiments as well.

What is claimed is:

1. A method of controlling a vehicle interior sterilization apparatus, the method comprising:
determining, by a control unit, whether a sterilization precondition for a vehicle is satisfied and a user sterilization request signal is received;
determining, by the control unit, whether a sterilization stability condition is satisfied, when the sterilization precondition is satisfied and the sterilization request signal is received;
determining, by the control unit, when the sterilization stability condition is satisfied, whether a battery operation condition is satisfied; and
performing, vehicle interior sterilization using the vehicle interior sterilization apparatus, when the battery operation condition is satisfied,
wherein performing the vehicle interior sterilization comprises opening a cover unit positioned to selectively cover an opening of a housing provided inside a roof lining and turning on a light source unit exposed to the interior of the vehicle.

2. The method of claim 1, wherein determining whether the sterilization precondition is satisfied comprises determining whether the vehicle is in an ignition-off state and a door is in a locked state after a passenger exits the vehicle, by the control unit.

3. The method of claim 1, wherein determining whether the sterilization stability condition is satisfied comprises determining that the sterilization stability condition is satisfied, when the following conditions are satisfied: the vehicle is stopped, a gear parking input is received, an window of the vehicle is closed, a door of the vehicle is locked, a smart key of the vehicle is outside, seat weight is not detected, and no object is detected in a rear seat of the vehicle, by the control unit.

4. The method of claim 3, wherein the determining whether the sterilization stability condition is satisfied further comprises terminating an operation when the sterilization stability condition is not satisfied.

5. The method of claim 1, wherein the determining whether the battery operation condition is satisfied comprises comparing a state of charge of a battery with a set value.

6. The method of claim 5, wherein the comparing the state of charge of the battery with the set value, comprises:
terminating an operation of the sterilization apparatus when the state of charge of the battery is equal to or lower than a first set value, an operation of the sterilization apparatus; and
setting an output-limit sterilization mode of the sterilization apparatus when the state of charge of the battery is greater than the first set value and is equal to or lower than a second set value.

7. The method of claim 1, wherein the performing the vehicle interior sterilization using the sterilization apparatus, comprises performing control, in a case where an interior temperature of the vehicle exceeds a first set temperature, for reducing an operating time of the sterilization apparatus or the amount of current applied to the light source unit, by the control unit.

8. The method of claim 1, wherein the performing the vehicle interior sterilization using the sterilization apparatus, comprises controlling the amount of current applied from the battery to each of the plurality of LEDs that form the light source unit, or controlling the number of lighting times of each of the plurality of LEDs, by the control unit.

9. The method of claim 8, wherein the control unit performs control for reducing the current applied to each of the LEDs positioned adjacent to the sterilization apparatus and increasing the current applied to each of the LEDs that emits light of the interior of the vehicle positioned relatively far from the sterilization apparatus.

10. The method of claim 1, wherein in the determining whether the sterilization precondition for the vehicle is satisfied and the user sterilization request signal is received,
the control unit determines, when the sterilization precondition is satisfied, whether the user sterilization request signal is received through an external manipulation unit.
